(19) United States
(12) Patent Application Publication
(10) Pub. No.: US 2012/0040301 A1
(43) Pub. Date: Feb. 16, 2012

(54) MANDIBULAR ADVANCEMENT

(75) Inventor: Joachim Ngiam, New South Wales (AU)

(73) Assignee: Joachim Nigiam, as trustee for Joachim Nigiam Trus Fund ABN, Homsby (AU)

(21) Appl. No.: 13/256,916

(22) PCT Filed: Mar. 23, 2010
(86) PCT No.: PCT/AU10/00334

§ 371 Date: Nov. 3, 2011

(30) Foreign Application Priority Data
Mar. 23, 2009 (AU) 2009901240

(51) Int. Cl. A61C 7/06; A61M 16/00

(52) U.S. Cl. 433/5; 433/24; 128/204.18

(57) ABSTRACT

There are provided various appliances for human mandibular advancement. One appliance comprises a frame having a first end adapted to be secured against a fixed part of the body of a subject; a second end equipped with a pulling platform; and an intervening length to present the pulling platform outside the mouth of the subject when the first end is mounted against the fixed part of the body of that subject. At least part of the intervening length of the frame is shaped to match the contours of the subject's face so that when the first end is secured against the fixed part of the body, and at least part of the intervening length lies against the matching contours of the subject's face. Other aspects include a dental device, method for advancing the mandible of a human subject, a shell, and a method for personalising the frame from a blank frame using the shell.

### CROSS-REFERENCE TO RELATED APPLICATION

The present application claims priority from Australian Provisional Patent Application No 2009901240 filed on 23 Mar. 2009, the content of which is incorporated herein by reference.

### TECHNICAL FIELD

This description generally concerns mandibular advancement, that is bringing forward of the lower jaw. Aspects of the invention include various appliances, a method for advancing the mandible of a human subject, a shell, a method for personalising the frame from a blank frame using the shell, a dental device for mandibular advancement and a method for advancing the mandible of a human subject using the dental device.

### BACKGROUND

Mandibular advancement is useful for treating a range of dental problems, for instance underdevelopment of the lower jaw. It is also useful for a number of medical problems, such as snoring obstructive sleep apnoea (OSA), which is a sleep disorder characterised by periodic reduction or cessation of breathing due to narrowing of the upper airway during sleep.

A range of oral appliances are currently available for mandibular advancement. These generally consist of respective ‘splints’ attached to each side of the maxilla (upper jaw) and each side of the mandible (lower jaw). The upper and lower splints interact with each other to draw the mandible forward. The interaction between the parts may rely on mechanical engagement, of for instance sloping surfaces of the opposed parts, springs, magnets or any combination of these things.

There are a number of disadvantages with using splints, such as restricting airflow, impeding speech and obstructing the tongue. A visit to the clinician is also generally necessary for adjustment. In addition, there will generally be some unwanted movement of the teeth caused by the reaction forces between the upper and lower jaw.

Maxillary advancement is also useful for a range of dental problems, particularly Class III malocclusions in children. This treatment typically makes use of a ‘facial mask’ that comprises a framework anchored to the forehead and chin, and a crossbar that is used to brace springs (elastic bands) attached to a pair of splints bonded to the teeth on respective sides of the upper jaw.

### SUMMARY

In a first aspect, there is provided a personalised appliance for human mandibular advancement, comprising a frame having
a first end adapted to be secured against a fixed part of the body of a subject,
a second end equipped with a pulling platform, and
an intervening length to present the pulling platform outside the mouth of the subject when the first end is mounted against the fixed part of the body of that subject. Wherein, at least part of the intervening length of the frame is shaped to match the contours of the subject's face so that when the first end is secured against the fixed part of the body, at least part of the intervening length lies against the matching contours of the subject's face.

Generally, the pulling platform is centred in front of the face and between the lips. In use, the pulling platform is attached to the subject's mandible by substantially inextensible means to pull it forward. Metal wires or cables (fishing line) may be suitable for this purpose.

Using this appliance to apply forward traction forces to a subject's mandible, no reciprocal force is exerted on the maxilla, thereby reducing unwanted side effects on the upper arch. Also, the bulk within the subject's mouth is reduced compared to ‘splints’ that are attached to each side of the maxilla (upper jaw) and each side of the mandible. Advantageously, this improves airflow and tongue posture, and reduces clockwise rotation of mandible and bite opening. The invention can be used to treat obstructive sleep apnoea, maxillary retrusion in skeletal Class III malocclusions, and potentially mandibular retrusion in the developing child to encourage mandibular translation or “bite jumping” and growth.

The first end of the frame may be secured against a fixed part of the skull, particularly by being strapped to the subject's forehead.

To assist in pulling the jaw forward, the pulling platform may include an adjustment mechanism that can be operated to vary the distance by which a subject's mandible is advanced.

The mechanism may be manual or motorised, in which case it may be computer controlled to vary the length of the attachment according to a preselected regime, or in response to stimuli. For instance, it may be programmed to pull the jaw further forward when snoring is detected, and to release after a period of quiet.

The attachment to the lower jaw may make use of temporary anchorage devices, such as orthodontic micro screws, or mini or micro implants that have no, or light, osseo-integration to bone. In this case, the temporary anchorage devices are implanted in the subject's lower jaw to provide skeletal anchorage for mandibular advancement.

Alternatively, the attachment to the jaw may be made to a dental prosthetic implant either directly or via an internal structure. In this case, the dental prosthetic implant also provides skeletal anchorage for mandibular advancement.

Alternatively again, the attachment to the jaw may be made to an appliance that is fixedly or removably secured to the teeth of the lower jaw. In this case, the appliance provides tooth-borne anchorage for mandibular advancement.

In a second aspect, there is provided an appliance for human mandibular or maxillary advancement, comprising a frame having
a first end adapted to be secured against a fixed part of the body of a subject,
a second end equipped with a pulling platform, and
an intervening length to present the pulling platform outside the mouth of the subject when the first end is secured against the fixed part of the body of that subject. Wherein, the pulling platform comprises an entirely extra-oral adjustment mechanism to control the distance by which the jaw is advanced when, in use, the mechanism is attached to the subject's jaw.

Advantageously, the mechanism is able to advance the mandible or maxilla with precision. For instance, the mandible or maxilla can be advanced at a minimum of ⅙ mm at a time, up to 70% of a subject's maximum jaw protrusion. The amount of advancement can also be readjusted as required by a clinician or the subject herself.

In a third aspect, there is provided an appliance for human mandibular advancement, comprising a frame to present a pulling platform extra orally centred in front of the face and between the lips of a subject, wherein the pulling platform is attached to the lower jaw to pull it forward.

In a fourth aspect, there is provided an appliance for human mandibular or maxillary advancement, comprising a frame to present a pulling platform extra orally centred in front of the face and between the lips of a subject, wherein the pulling platform is attached to the jaw by substantially inextensible means to pull it forward.

In a fifth aspect, there is provided a method for advancing the mandible of a human subject, comprising the steps of:
locating a pulling platform in a fixed position relative to the subject's head outside the subject's mouth and centred in front of the face and between the lips; and
attaching the pulling platform to the subject's mandible using substantially inextensible means.

The substantially inextensible means may be metal wires or cables (fishing line) extending from the pulling platform to the subject's mandible.

In a sixth aspect, there is provided a shell for forming a negative mould of a subject's face, comprising a plasticly deformable skeleton having multiple regions extending from a web that extends around a central breathing hole, all encapsulated within an elastic material such that, in use, the shell can be deformed into approximate conformity with the subject's face.

The skeleton may be made from metal mesh. The elastic material may be silicone. The shell may further comprise an integrated layer of impression material that can be activated and deactivated to make an impression of a subject's face. The integrated layer may be made of thermoplastic material, in which case activation is by way of heat.

In a seventh aspect, there is provided a method of personalising, from a blank frame, a frame that conforms to the contours of a subject's face, along at least part of its length, the method comprising:
using a shell according to the sixth aspect to form a negative mould of the subject's face; and
matching at least part of the length of the blank frame to the contours of the subject's face using the negative mould, or a positive cast made from the negative mould.

In an eighth aspect, there is provided a dental device for human mandibular advancement, comprising:
an elongated portion having first and second ends, the portion having a variable length and provided with a resilient member,
a locking mechanism to retain the elongated portion at a desired length, and
an attachment formation on each end of the elongated portion, each attachment formation being shaped and sized for connection with an anchorage device implantable in a subject's mandible or maxilla,
wherein, in use, the elongated portion is retained by the locking mechanism at a desired length and engaged with anchorage devices implanted in the subject's mandible and maxilla via the attachment formations such that the resilient member is operable to urge the subject's mandible forward relative to the maxilla.

The attachment formations may be shaped and sized for click connection with the anchorage device.

The attachment formations may be each provided with a ring surrounding a gripping portion for connection with a ring indentation and a head of the anchorage device respectively.

The elongated portion may comprise inner and outer telescopic members that are movable relative to each other and the locking mechanism is a screw that retains the members in a fixed relationship.

In a ninth aspect, there is provided a method for advancing the mandible of a human subject using the dental device according to the eighth aspect, the method comprising
adjusting the length of the elongated portion and retaining the elongated portion at a desired length using the locking formation; and
connecting ends of the elongated portion to a respective anchorage device implanted on the subject's mandible or maxilla via the corresponding attachment formation.

### BRIEF DESCRIPTION OF THE DRAWINGS

The invention will now be described by way of non-limiting examples with reference to the accompanying drawings, in which:

FIG. 1 is a frontal view of a first example of an appliance exemplifying the invention.

FIG. 2 is a side view of the appliance shown in FIG. 1.

FIG. 3 is a pictorial view of a substantially inextensible means.

FIG. 4(a) is a pictorial view of the substantially inextensible means in FIG. 3 when attached to a subject's mandible using temporary anchorage devices.

FIG. 4(b) is a side view of a temporary anchorage device.

FIG. 4(c) is a pictorial view of the substantially inextensible means in FIG. 3 when attached to a subject's mandible using dental prosthetic implants.

FIG. 4(d) is a pictorial view of the substantially inextensible means in FIG. 3 when attached to a subject's mandible using an appliance attached to the teeth of the lower jaw.

FIG. 5 is a flowchart of a method of advancing the mandible of a human subject.

FIG. 6 is a flowchart of a method of fabricating a frame that conforms to the contours of a subjects face.

FIG. 7 is an exploded view of a shell.

FIG. 8(a) is a pictorial view of a cast for fabricating a shell.

FIG. 8(b) is a pictorial view of the cast in FIG. 8(a), after a layer of silicone is poured onto a skeleton.

FIG. 8(c) is a pictorial view of a shell fabricated using the cast in FIG. 8(a).

FIG. 9(a) is a pictorial view of a shell, with a layer of impression material applied onto its inner surface.

FIG. 9(b) is a pictorial view of a subject using the shell in FIG. 9(a).

FIG. 9(c) is a pictorial view of the shell in FIG. 9(b) after a negative mould of the subject's face is made.

FIG. 10(a) is a pictorial view of the shell in FIG. 9(c), after dental stone is poured into the shell to make a positive cast.

FIG. 10(b) is a pictorial view of positive cast created in FIG. 10(a).

FIG. 10(c) is a pictorial view of the shell in FIG. 10(a), after the positive cast and negative mould are removed.

FIG. 11(a) is a frontal view of a second example of the appliance in FIG. 1.

FIG. 11(b) is a frontal view of a third example of the appliance in FIG. 1.

FIG. 12(a) is a pictorial view of a dental device for mandibular advancement when attached to a subject's maxilla and mandible via two anchorage devices.

FIG. 12(b) is a pictorial view of the dental device.

FIG. 12(c) is a cross-sectional pictorial view of the anchorage device in FIG. 12(a) when engaged with the dental device in FIG. 12(b).

### BEST MODES OF THE INVENTION

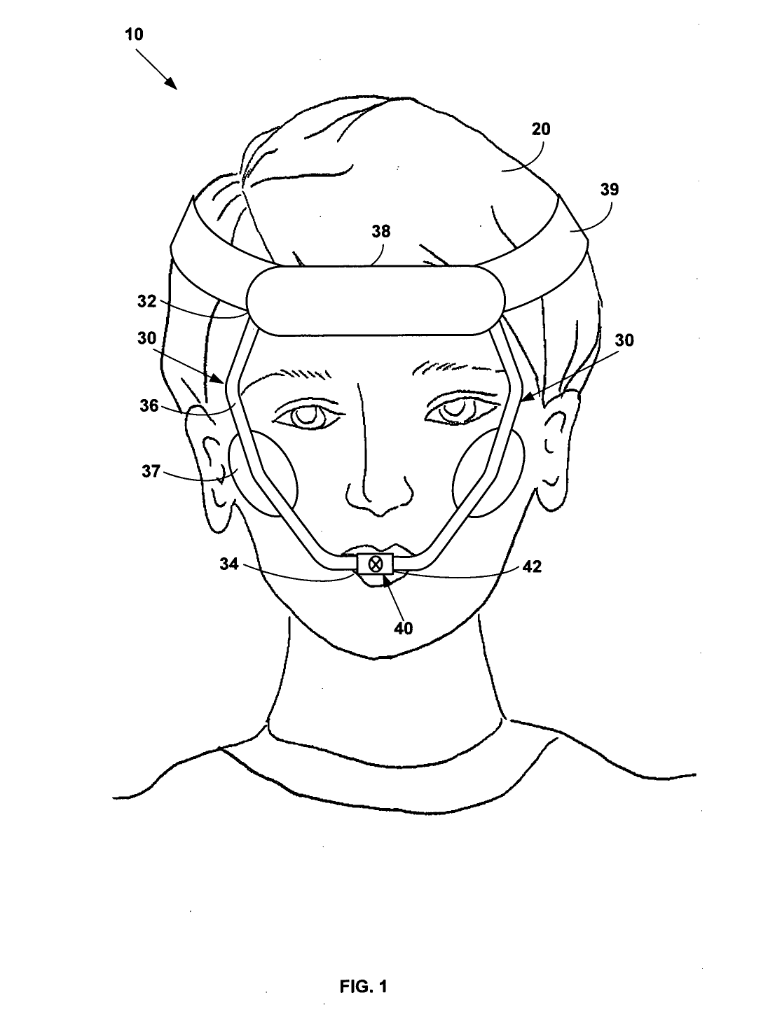

Referring first to FIG. 1, the appliance 10 exemplifying the invention is shown worn by a subject 20 requiring mandibular or maxillary advancement. The appliance 10 comprises two identical frames 30, each having:
A first end 32 to be secured against a fixed part of the body of a subject 20, specifically against the upper part of the subject's skull.
A second end 34 equipped with a pulling platform 40. And,
An intervening length 36 extending from the first end 32 to the second end 34 to present the pulling platform 40 outside the mouth of the subject 20 when the first end 32 is secured against the upper part of that subject's 20 skull.

The appliance 10 is a laboratory-fabricated device that is personalised for each subject 20. In particular, at least part of the intervening length 36 of the frame 30 is shaped to match the contours of the subject's 20 face so that when the first end is secured against the fixed part of the body, at least part of the intervening length 36 lies against the matching contours of the subject's 20 face.

In the example shown in FIG. 1, the intervening length 36 is shaped to match the contours of the subject's face along the right hand side of the subject's forehead, cheekbone and cheek so as to present the pulling platform 40 outside the centre of the mouth. A zygomatic pad 37 is placed below one section of the intervening length 36 to make the appliance 10 more comfortable.

The first end 32 of each frame 30 is secured against the upper part of the subject's skull by being strapped to the subject's forehead using strap 39. The first end 32 is attached to a laboratory-fabricated frontal plate 38 that has been shaped to match contours of the subject's forehead so as to better secure the frame 30 against the subject's forehead. The frontal plate 38 is made acrylic, but other suitable material such as plastics may be used. Strap 39 is elastic to allow stretching to fit around the subject's head. Alternatively, strap 39 may be inelastic and fastened using hook and loop fasteners such as Velcro™.

Pulling Platform 40

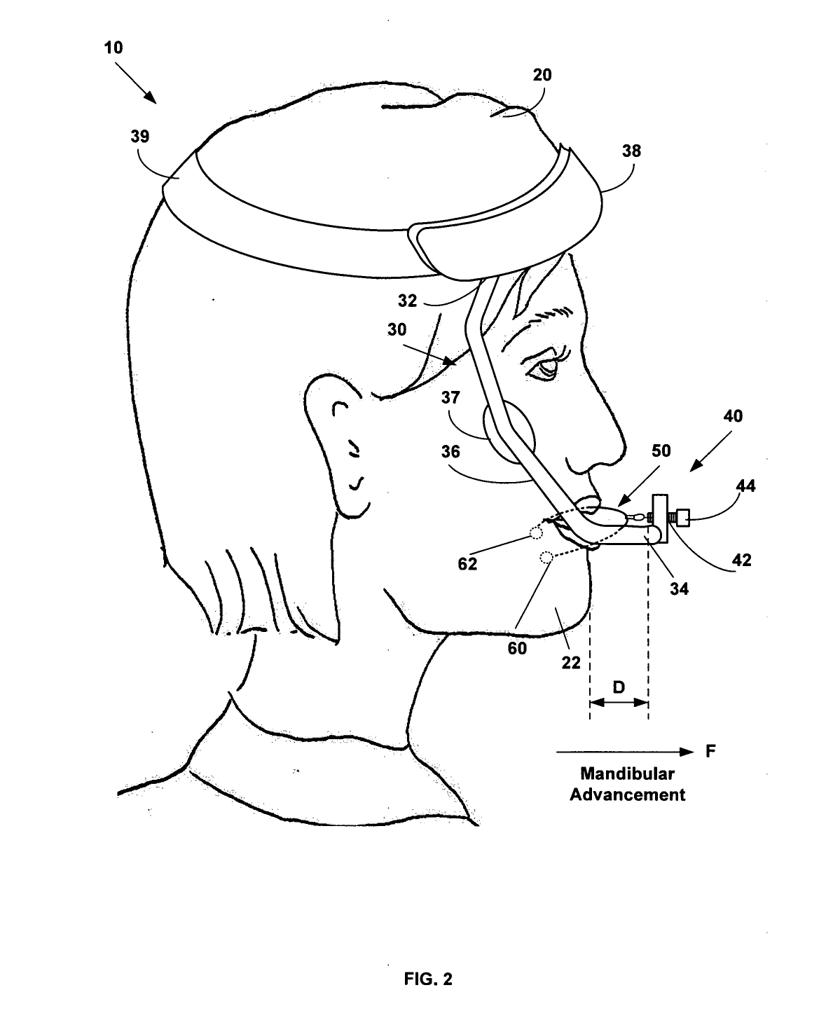

Referring also to FIG. 2, the second end 34 of the frames 30 is equipped with the pulling platform 40, which is centred in front of the subject's face and between the lips when the first end 32 is strapped to the subject's head.

The pulling platform 40 comprises an entirely extra-oral adjustment mechanism 42 which can be operated to vary the distance by which a subject's mandible 22 is advanced. The adjustment mechanism shown 42 is in the form of a manually operatable screw (also 42) having a turning knob 44. The screw 42 is attached to the subject's mandible 22 using a substantially inextensible means 50. In use, the substantially inextensible means 50 is attached to the subject's mandible 22 using anchorage devices 60 and 62.

When the turning knob 44 is rotated in a specified direction, the screw 42 will move away from the subject's face, pulling the attached substantially inextensible means 50, and therefore the subject's mandible 22, towards the forward direction (labelled ‘F’). The screw 42 may be chosen such that one full turn of the knob 44 advances the subject's mandible 22 by at a minimum of, for instance, 1 mm. The length of the screw 42 may be also chosen such that the subject's mandible can only be advanced up to a certain percentage of the subject's maximum mandibular protrusion. In practice, the maximum displacement is generally 70% of the subject's maximum protrusion.

Advantageously, the extra-oral adjustment mechanism 42 allows a clinician to vary the mandible displacement of a subject, for instance during a polysomnographic study to determine an effective mandibular displacement to treat obstructive sleep apnoea. Further self-readjustments can be made by the subject whenever the displacement set by the clinician is no longer effective, for instance due to muscle relaxation or adaptation after prolonged use.

Although not shown, the adjustment mechanism 42 may also be motorised. In this case, the mechanism 42 may be computer-controlled to displace a subject's mandible 22 to a preselected regime, or in response to stimuli detected by a sensor. For instance, it may be programmed to pull the mandible 22 forward when snoring is detected by the sensor, and to release after a period of quiet. Further, the motorised adjustment mechanism 42 may comprise a remote control operable to send a radio signal to the adjustment mechanism 42 to increase or decrease the displacement.

Substantially Inextensible Means 50

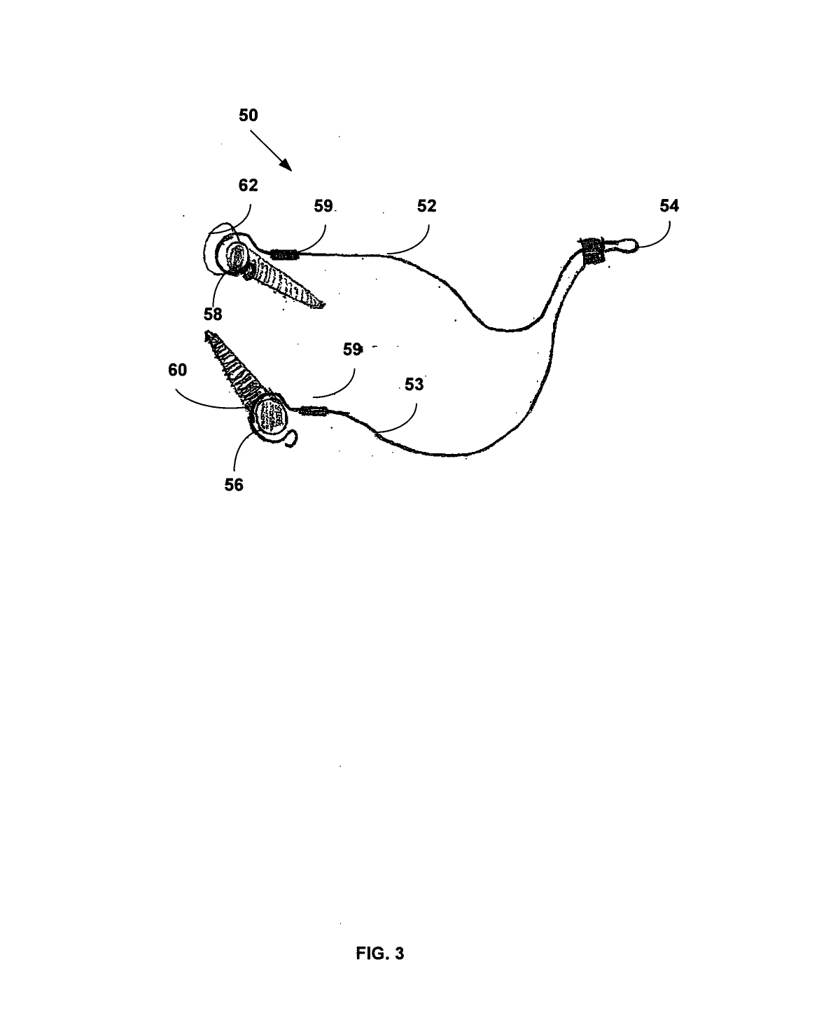

Referring now to FIG. 3, the substantially inextensible means 50 attaches the extra-oral pulling platform 40 centred outside the subject's mouth to the subject's (intra-oral) mandible 22 for mandibular advancement. The substantially inextensible means 50 may be made of metal wires, cables or plastics such as fishing lines. Unlike elastomeric banding devices, the substantially inextensible means 50 do not degrade with oral fluids.

The substantially inextensible means 50 comprises two arms 52 and 53, to which hooks 56 and 58 are attached. A hoop 54 is formed on the opposite end of the substantially inextensible means 50. The hooks 56 and 58 are attached to the elongated member 52 using crimping or welding at 59. In use, the hooks 56 and 58 are each attached to a corresponding intra-oral anchorage devices 60 (62) held in fixed relationship to the subject's mandible. On the opposite end, the hoop 54 is attached to an extra-oral pulling platform 40, such as to a hook (not shown) extending from the pulling platform 40.

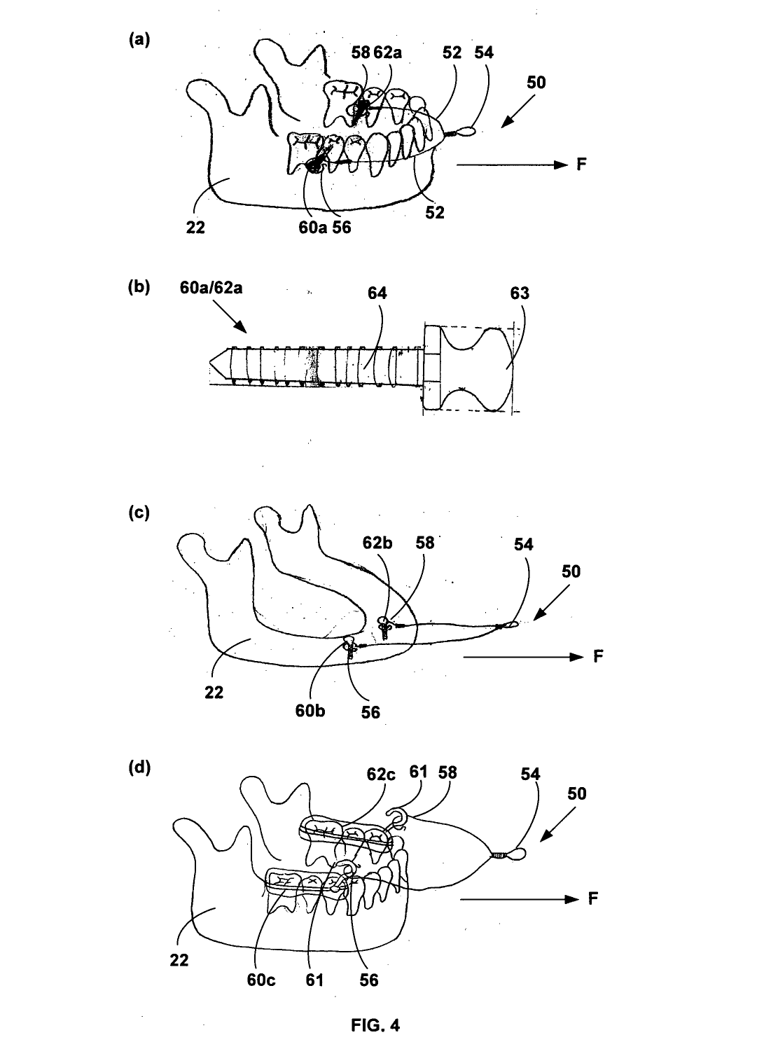

The substantially inextensible means 50 is adaptable in order to connect the extra-oral pulling platform 40 to a variety of intra-oral anchorage devices 60 and 62. Referring now to FIGS. 4(a), 4(b), 4(c) and 4(d), the substantially inextensible means 50 is designed to be attached to one or more of the following anchorage devices:

(a) Temporary anchorage devices.

(b) Dental prosthetic implants.

(c) An appliance that is fixedly or removably secured to the teeth on the mandible 22.

First in FIG. 4(a), the temporary anchorage devices 60a and 62a are orthodontic micro screws, or mini or micro implants that provide skeletal anchorage to the mandible 22. These temporary mini-implants have no, or light, osseo-integration and therefore are easily implanted and removed. Generally, insertion of these temporary anchorage devices 60a and 62a takes only a few minutes. Their removal can, generally, be performed with relative ease and little discomfort to the patient. They are considerable smaller in diameter compared to conventional dental implants and can be placed in between roots of teeth and in edentulous spans where there are no teeth.

To provide skeletal anchorage, one temporary anchorage device 60a (62a) is implanted on each side of the subject's mandible 22. The temporary anchorage devices 60a and 62a are shaped and sized to engage with the substantially inextensible means 50. As shown more clearly in FIG. 4(b), each temporary anchorage device 60 (62) has a mushroom-shaped head 63 for engagement with a hook 56 (58) of the substantially inextensible means 50, and a threaded shaft 64 for implantation into the subject's mandible 22. The elongated arms 52 and 53 of the substantially inextensible means 50 extend along the side of the teeth on the mandible 22 to meet at the hoop 54 for attachment to a pulling platform 40.

With reference to FIG. 4(c), dental implants 60b and 62b are designed to be osseo-integrated to the subject's bone and therefore are immobile and resistant to movement. As such, the anchorage to the subject's mandible 22 provided by the osseo-integrated dental implants is also a form of skeletal anchorage. In use, the substantially inextensible means 50 is attached to the dental implants 60b and 62b via hooks 56 and 58 respectively to provide an anterior pulling force for mandibular advancement.

The process of placing an osseo-integrated implant is much more involved than that of a temporary anchorage device. Typically, a healing period is recommended and once the implants are osseo-integrated (stage 1). Then, a prosthetic stage (stage 2) is implemented where a tooth coloured crown/bridge is attached to the implants in stage 1. For patients who are completely edentulous (no teeth) on the lower arch, a lower denture is fabricated to give the patient “teeth” in the lower jaw. This denture is only supported by soft tissue and the denture tends to move and “float” between the tongue and cheek muscles.

To provide better stability, sometimes, two dental prosthetic implants are placed in the bone in the lower jaw. To these two dental osseo-integrated implants, a super structure called an abutment arising from the implant above the gum tissue is often fixed. These abutments connect to the denture to provide increase stability and retention for a lower denture (overdenture). These dental implants can provide support and retention for an overdenture during the day, and can be connected to the pulling platform 40 for mandibular advancement after the denture is removed at night.

Referring now to FIG. 4(d), the substantially inextensible means 60 can also be connected to lower appliances 60c and 62c that are fixedly or removably secured to the teeth on the mandible 22 to provide tooth-borne anchorage. Unlike attachment via temporary anchorage devices (60a, 62a) and dental prosthetic implants (60b, 62b), the lower appliances 60c and 62c are not suitable for edentulous patients because they usually require a minimum usually of 6 to 10 teeth to be present.

The appliances 60c and 62c can be made of a combination of metal and acrylic. In use, the hooks 56 and 58 of the substantially inextensible means 50 are each engaged with a corresponding attachment member 61, also in the form of a hook, of the appliances 60c and 62c.

Method for Mandibular Advancement Using Appliance 10

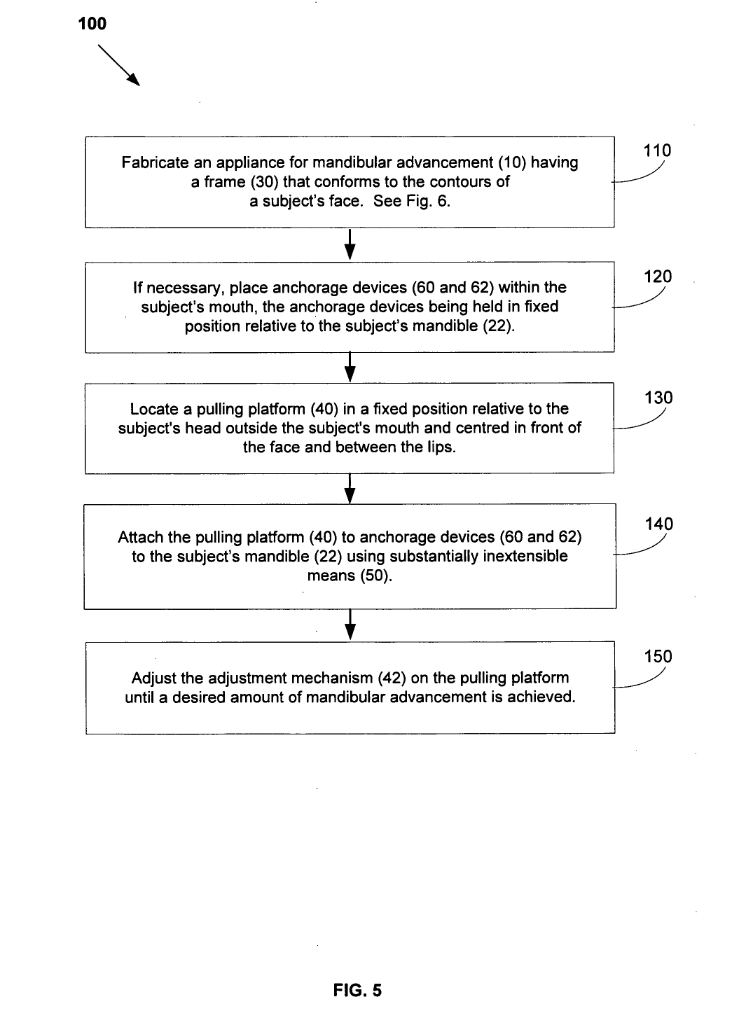

Referring to FIG. 5, a method for advancing the mandible of a human subject using the appliance 10 will now be described. The process typically starts with a subject 20 going to a clinician for treatment of, for instance, obstructive sleep apnoea.

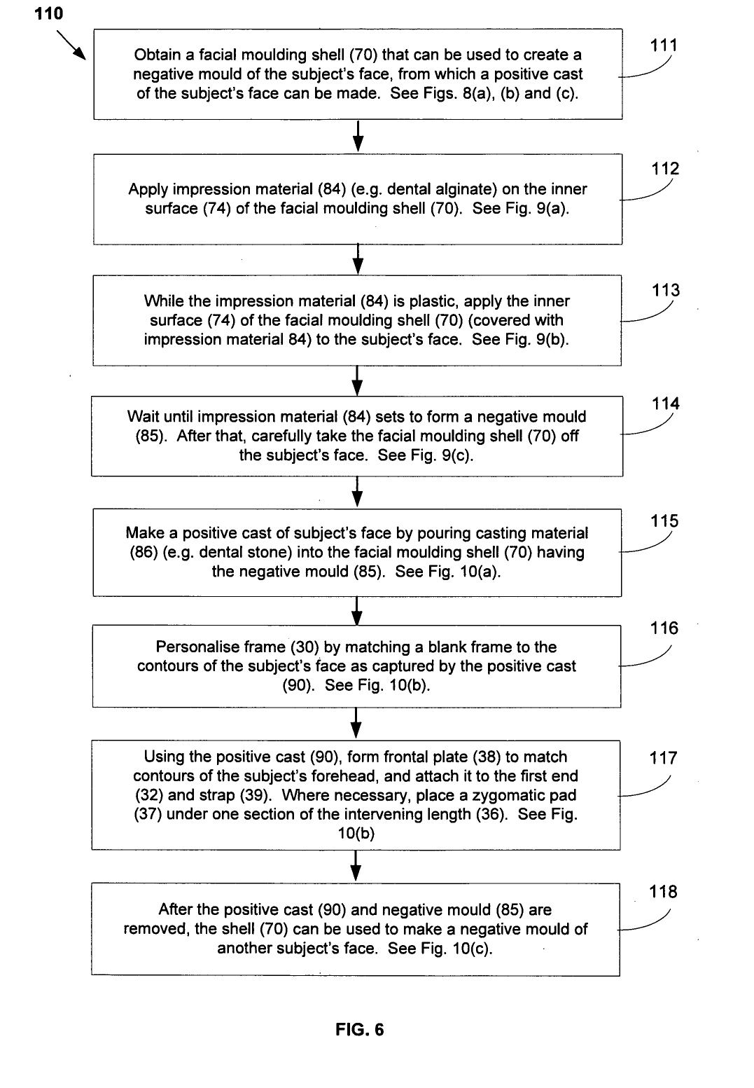

In step 110, a personalised appliance 10 having a frame 30 that conforms to the contours of the subject's face is fabricated using the method outlined in FIG. 6. Personalising the frame 30 allows the appliance 10 to be worn more comfortably by the subject 20, taking into account the subject's face contours and sleeping habits. If the subject 20 does not have any existing intra-oral anchorage devices (60 and 62) that can provide anchorage to the subject's mandible 22, these anchorage devices need to be implanted or fitted in step 120.

Once custom-made, the subject 20 can then use the appliance for mandibular advancement. Specifically in step 130, the pulling platform 40 at the second end 34 of the frame 30 is located in a fixed position relative to the subject's head and outside the subject's mouth and centred in front of the face and between the lips. This is achieved by first placing the frontal plate 38 of the frame 30 against the subject's forehead and then fastening the strap 39 extending from the frontal plate 38 around the subject's head.

The pulling platform 40 is then attached to the subject's mandible using the substantially inextensible means 50; see step 140. This is achieved by first engaging the hooks 56 and 58 of the substantially inextensible means 50 with the corresponding anchorage devices 60 and 62 that are held in fixed position relative to the subject's mandible 22. The hoop end 54 of the substantially inextensible means 50 is then connected to adjustment mechanism 42 of the pulling platform 40.

The distance (“D” in FIG. 1) by which the subject's mandible 22 is displaced (in the forward direction “F” in FIG. 1) is pre-set by a clinician. However, due to muscle relaxation over time, the distance required may need re-adjusted. In that case, the subject or a third person can vary the distance from time to time using the adjustment mechanism 42.

Method of Fabricating Frame 30

Referring now to FIG. 6, a method of fabricating a frame 30 that conforms to the contours of the subject's face will now be described.

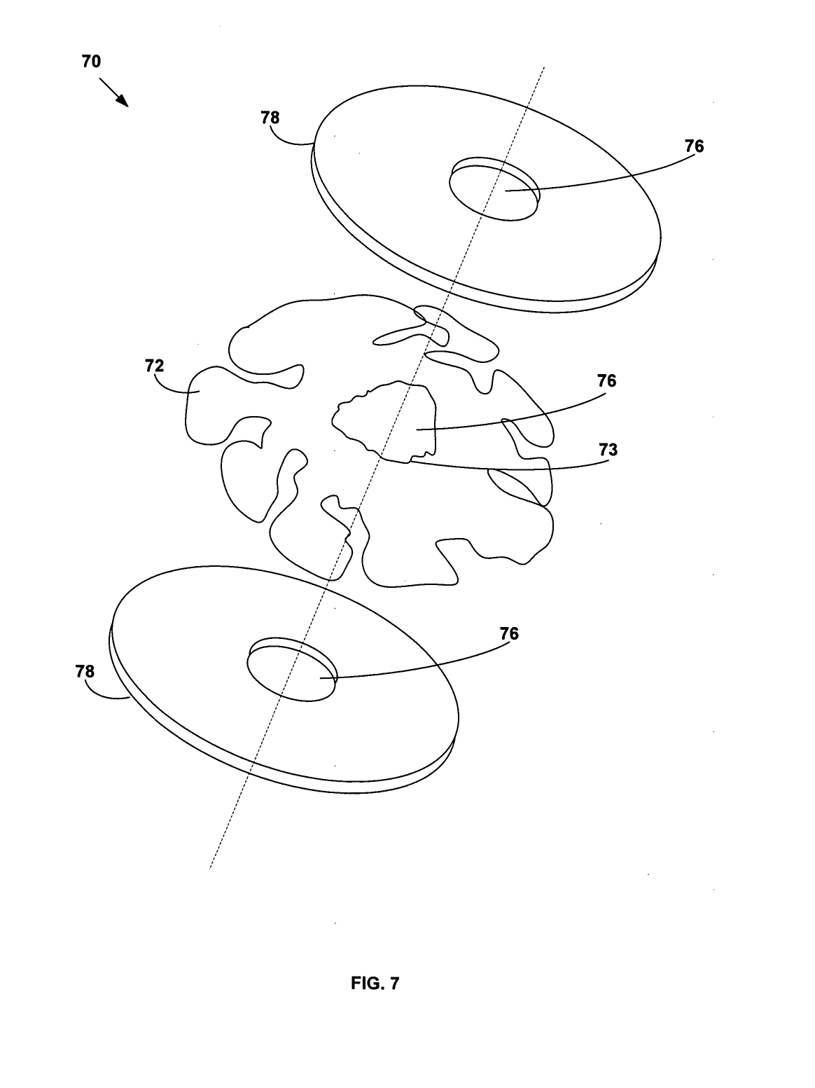

A facial moulding shell 70 is first used to create a negative mould of a subject's face; see FIG. 111. As shown in the exploded view in FIG. 7, the shell 70 comprises a plasticly deformable skeleton 72 encapsulated in a silicone body having inner 74 and outer 78 layers. The skeleton 72 is in the form of a piece metal mesh having multiple regions with a web 73 that extends around a central breathing hole 76, such that, in use, the shell 70 is deformable into approximate conformity with a subject's face.

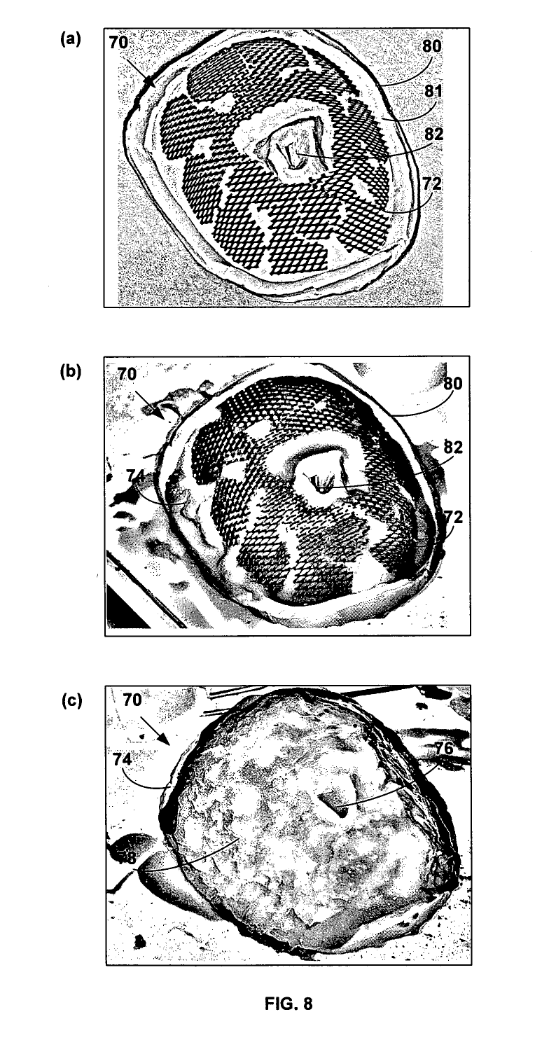

The process of making the shell 70 using a shell mould 80 is illustrated in FIGS. 8(a), (b) and (c). Specifically, a skeleton 72 is first placed on the shell mould 80, which in this case, is a positive face cast of a person (not of the subject 20); see FIG. 8(a). The body of the shell mould 80 is generally made of dental stone while its periphery is made of clay that is shaped to form a receptacle to hold the skeleton 72 and silicone forming the body. Of course, other plastic material such as Plaster of Paris may be used to make the shell mould 80.

Then, in FIG. 8(b), a first layer of liquid silicone is poured onto the skeleton 72 on the shell mould 80 to form the inner surface 74. After the first layer sets, more silicone is poured onto the first layer. This step of silicone pouring is repeated until the skeleton 72 is fully encapsulated within the silicone.

The product of this process is the shell 70 shown in FIG. 8(c). The shell 70 has an inner surface 74 that follows the shape of the mould 80, an outer surface 78 having rough texture due to the layers of silicone encapsulating the skeleton 72, and a central breathing hole 76. The facial moulding shell 70 fabricated using this process is deformable, and therefore reusable to take an impression of the face of a plurality of subjects.

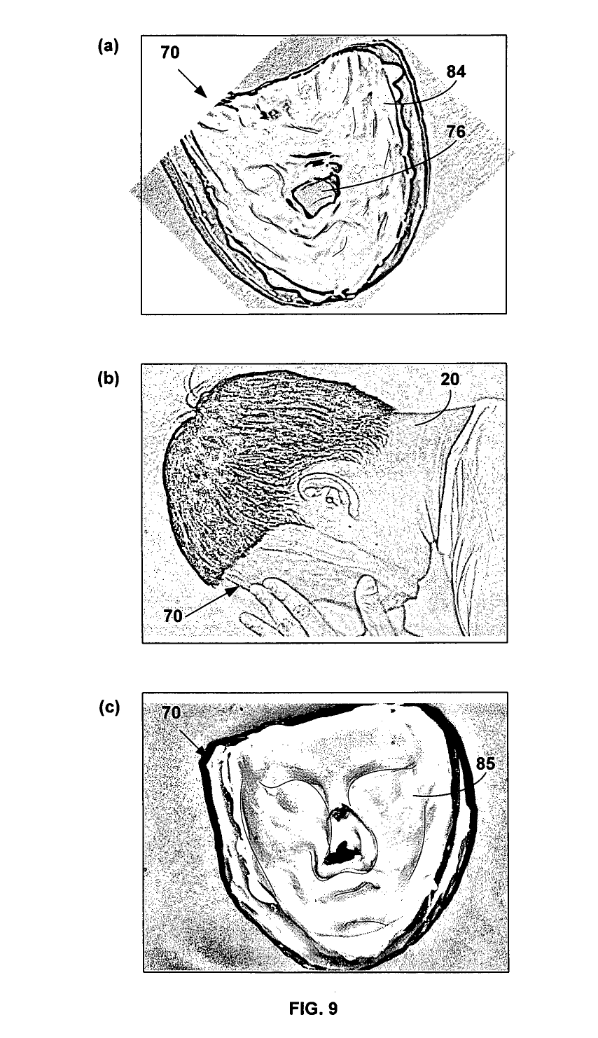

To use the shell 70 to make a negative mould of a subject's face, an impression material 84 is applied onto the inner surface 74 of the shell 70, carefully avoiding the central breathing hole 76; see step 112 in FIG. 7 and FIG. 9(a). For instance, the impression material 84 may be a dental alginate mixture prepared by mixing water with alginate powder. Then, while the impression material 84 is still plastic, the inner surface 74 of the shell 70 is pressed against the subject's face, and held in this position until the impression material 84 sets; see step 113 in FIG. 7 and FIG. 9(b). The subject's face, especially areas with facial hair, could first be given a light covering of petroleum jelly to ease removal of impression material 84. As impression material 84 such as dental alginate usually sets within 2 to 3 minutes, this method allows a negative mould of a subject's face to be taken quickly at a clinic.

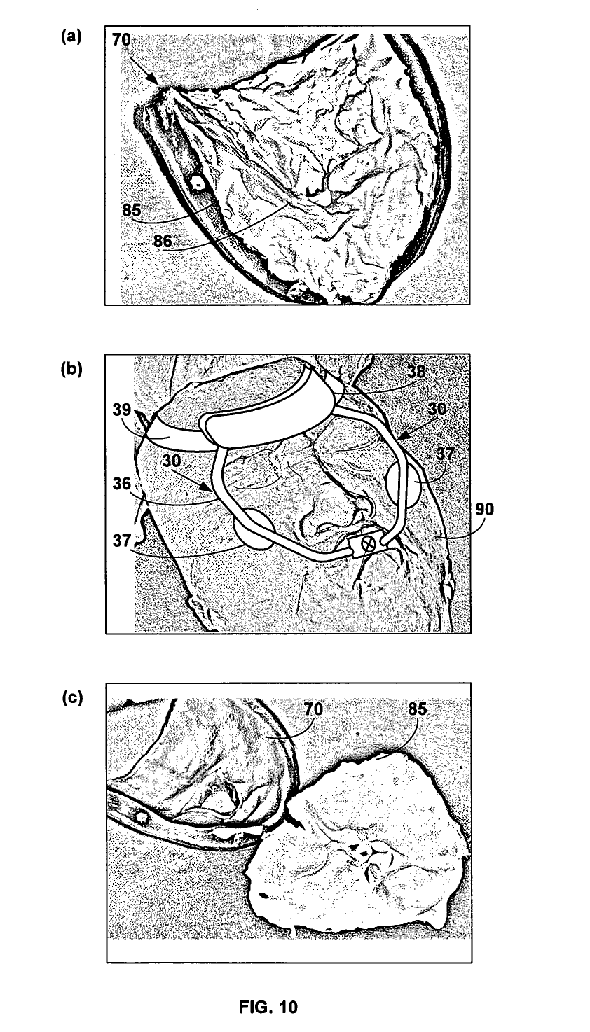

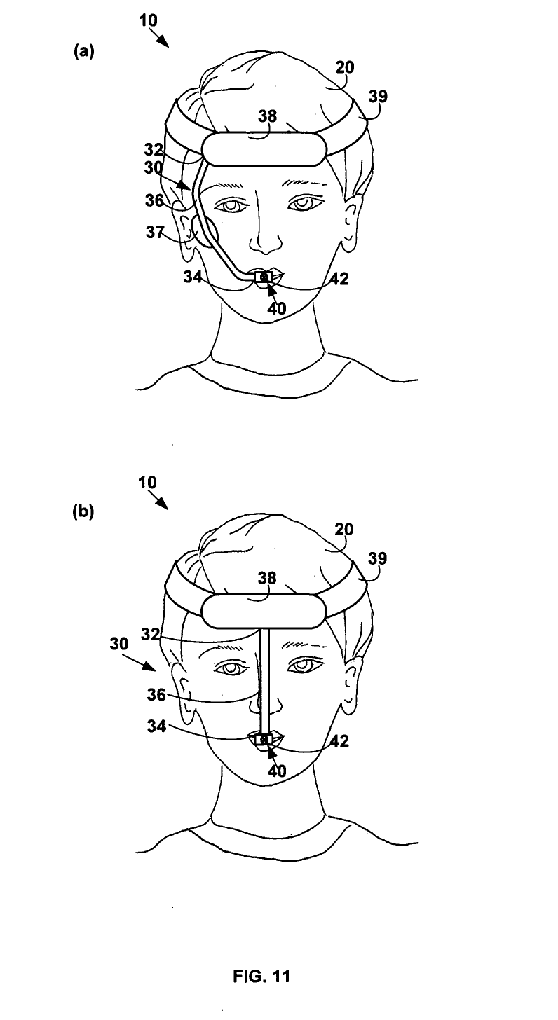

Finally, after the impression material 84 in FIG. 9(a) sets to form a negative mould 85 of the subject's face, the shell 70, together with the negative mould 85, is then carefully removed from the subject's face; see step 114 in FIG. 7. As shown in FIG. 9(c), the negative mould 85 registers the contours of the subject's face. The negative mould 85 can then be used to create a positive face cast. This involves pouring casting material 86 such as dental stone or Plaster of Paris into the shell 70; see step 115 in FIG. 7 and FIG. 10(a). Once dried, the casting material 86 is removed to obtain the positive cast 90 in FIG. 10(b).

Next, frames 30 that conform to the contours of both sides of the subject's face, as captured by the positive cast 90, can be formed from a blank, unshaped frame (not shown); step 116 in FIG. 7 and FIG. 10(b). The blank frame may be made of material such as plastics or metal wires that can be matched, at least part of its length, to the contours of the subject's face. Also, the blank frame may be fabricated by a clinician or pre-fabricated elsewhere. This method of personalising the frames 30 takes into account of the shape of the subject's forehead, cheekbone and cheek, as well as the position of the subject's mouth. Where appropriate, a zygomatic pad 37 can be placed under one section of the intervening length 36 of the frames 30 to improve comfort.

Then in step 117 in FIG. 7, a frontal plate 38 is fabricated to conform to the contours of the subject's forehead and connected to the first end 32 of the frame 30 and strap 39. The frontal plate 38 may be made of plastics or acrylic. Once formed, the pulling platform 40 should be positioned outside the mouth of the subject 20 when the first end 32 is secured against the upper part of that subject's 20 skull. After the positive cast 90 and negative mould 85 are removed, the facial moulding shell can be reused to make a positive cast of another subject; see step 118 in FIG. 7 and FIG. 10(c).

Alternatively, the frame 30 can be formed to match the contours of the subject's face without forming the positive cast (90). Instead, the negative mould 85 of the subject's face in FIG. 9 is used for this purpose. Specifically, like in steps 116 and 117 in FIG. 7, this involves bending at least part of the intervening length 36 of the frame 30 to match the contours registered by the negative mould 85. One or more zygomatic pads 37 can be placed under a section of the intervening length 36 to improve comfort.

Although the invention has been described with reference to a particular example, it will be appreciated by the appropriately skilled person that many modifications and additions are possible. For instance, the appliance 10 can also be used for maxillary advancement by connecting the pulling platform 40 to the subject's maxilla. In this case, anchorage devices 60 and 62 also need to be on the maxilla.

Also, while the frames 30 have been shown in FIG. 1 to extend from the subject's forehead to both the left and right hand sides of the subject's face, the appliance 10 may be modified to only have one frame that extends from either the right or the left hand side of the subject's face; see FIG. 10(a). Alternatively, the appliance 10 may have one frame that extends from the subject's forehead to the middle of the face (i.e. mid-sagittal plane); see FIG. 10(b). This allows subjects to sleep on their preferred left or right side.

Also, it is envisaged that the facial moulding shell 70 can be modified to include an integrated layer of impression material that can be activated and deactivated to make an impression of a subject's face. The impression material may be made of thermoplastic material, in which case activation is by way of heat. This way, step 112 in FIG. 7 (i.e. applying a layer on the inner surface of the shell 70) can be skipped.

Outside of the field of dentistry, it is envisaged that the facial moulding shell 70 could be used in the following fields:

(a) Medical field, to create a full or partial face cast for prosthetic surgery

(b) Optometry, to create a personalised pair of spectacles or eye mask;

(c) Forensics, to create a face cast of a crime victim;

(d) Fabrication of breathing apparatus such as customised CPAP masks, diver breathing apparatus and full face masks.

(e) Cosmetic and beauty industry, such as to create personalised beauty masks.

Also, the negative mould 85 removed from the shell 70 in step 118 in FIG. 7 can be used for other purposes. For instance, the negative mould can be used as a face mask, onto which special effects make-up can be applied.

Although not illustrated, the appliance 10 may have an integrated continuous positive airway pressure (CPAP) mask. The subject is connected to a positive pressure air supply via a nose mask or nasal prong that is integrated with the appliance 10. In use, the combination of mandibular advancement using the appliance 10 and provision of a positive pressure air supply using a CPAP mask keeps the subject's upper airway open during sleep to reduce the likelihood of upper airway occlusions.

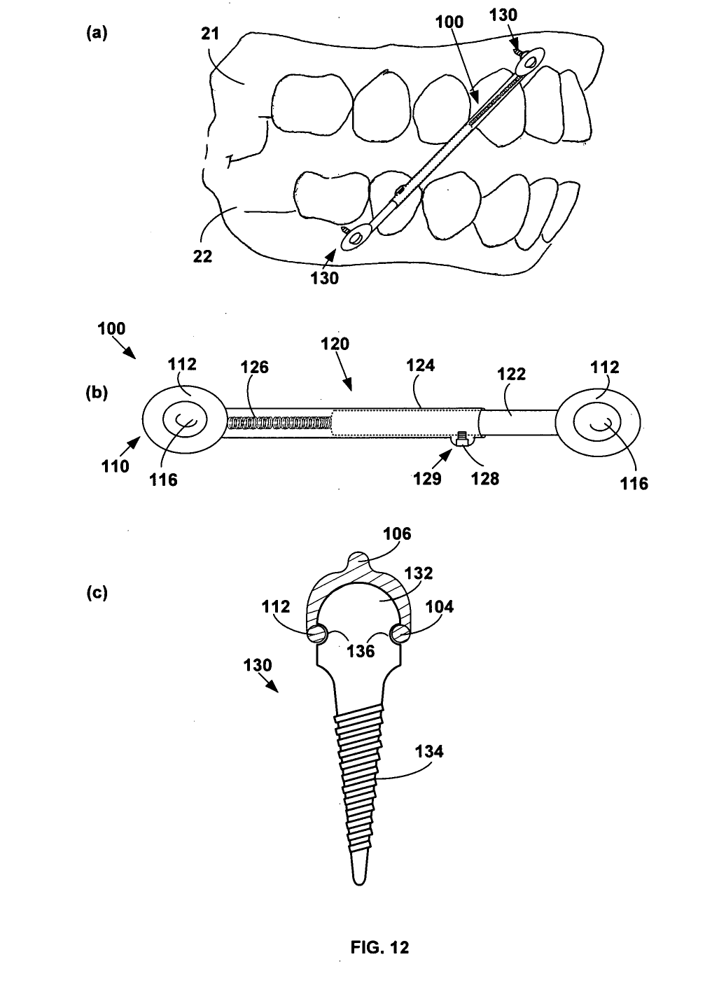

The treatment of sleep apnoea can also be enhanced using a dental device 100 for mandibular advancement, such as when the appliance 10 is not in use. As shown in FIG. 12(a), the dental device 100 is attached to two anchorage devices 130 respectively implanted in the subject's maxilla 21 and mandible 22 to urge the mandible 22 forward. Although only the right side of the mouth is shown in FIG. 12(a) for simplicity, an identical dental device 100 is also placed on the left side.

Referring also to FIG. 12(b), the dental device 100 comprises an elongated portion 120 having a variable length, a locking mechanism 128 to retain the elongated portion 120 at a desired length, and an attachment formation 110 on each end of the elongated portion 120 for attachment with a respective anchorage device 130.

The length of the elongated portion 120 is adjustable by moving an inner telescopic member 122 within an outer telescopic member 124. To fix the elongated portion at a desired length, the inner 122 and outer 124 telescopic members are retained in a fixed relationship relative to each other using the locking mechanism 128.

The locking mechanism 128 is in the form of a hex screw housed within the outer 124 telescopic member, but its head is accessible externally via an opening 129 in the member 124 for easy manipulation. The tip of the hex screw 128 engages the inner telescopic member 122 in a friction fit engagement to prevent movement of the outer telescopic member 124 with respect to the inner telescopic member 122. The resilient member 126 within the outer telescopic member 124 helps retain this engagement while exerting a forward force onto the subject's mandible 22 to urge it forward relative to the maxilla 21.

The attachment formations 110 are each shaped and sized for attachment with an anchorage device 130 implanted in the subject's mandible 22 or maxilla 21. Each attachment formation 110 comprises a continuous ring 112 surrounding a central gripping portion 116. As shown more clearly in FIG. 12(c), the gripping portion 116 has a small protrusion that is designed for gripping by a subject while locating an anchoring device 130 within the subject's mouth.

The anchorage device 130 is generally in the form of a micro screw, mini screw or dental prosthetic implant. The anchorage device 130 comprises a mushroom-shaped head 132 that is shaped and sized for a “snap” or “click” connection with the attachment formation 110, and a threaded shaft 134 for implantation in the subject's maxilla 21 and mandible 22. The mushroom-shaped head 132 is formed with a ring indentation 136 for retaining the continuous ring 112 of the attachment formation 110.

When in use, the length of the elongated portion 120 is determined and adjusted by a clinician by moving the inner telescopic member 122 within the outer telescopic member 124 until a desired length is achieved. The telescopic members 122 124 are then retained by screwing the hex screw 128 into the inner telescopic member 122 through the opening in the outer telescopic member 124. The suitable length depends on the amount of mandibular advancement required by the subject.

To attach the attachment formations 110 to the anchorage device 130, the gripping portion 116 at one end of the device 100 is first held by one or two fingers and then “clicked” onto the mushroom-shaped head 132 of an anchorage device 130 within the subject's mouth. When connected, the continuous ring 112 of the attachment formation 110 is fitted within a corresponding ring indentation 136 in the mushroom-shaped head 132; see FIG. 12(c).

The process is repeated for the other attachment formation 110 of the dental device 100 such that both formations 110 are connected to a respective anchorage device 130 implanted in the subject's mandible 22 or maxilla 21. The same is repeated for the other side of the subject's mouth such that two dental devices 100 are used at the same time.

The use of a dental devices 100 on both sides of the subject's mouth helps to advance the subject's mandible forward and keep the subject's upper airway open during sleep. This reduces the likelihood of upper airway occlusions and therefore sleep apnoea. The attachment formations 110 and telescopic members 122 and 124 can be made of suitable material such as silicon.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

For example, the ring 112 of the attachment formation 110 may not be continuous at all. A hook-like design can be used for the ring 112, as long as engagement with the corresponding indentation 136 in the anchorage device 130 can be achieved.

1. A personalised appliance for human mandibular advancement, comprising a frame having:
a first end adapted to be secured against a fixed part of the body of a subject;
a second end equipped with a pulling platform; and
an intervening length to present the pulling platform outside the mouth of the subject when the first end is mounted against the fixed part of the body of that subject; wherein, at least part of the intervening length of the frame is shaped to match the contours of the subject's face so that when the first end is secured against the fixed part of the body, at least part of the intervening length lies against the matching contours of the subject's face.

2. An appliance according to claim 1, wherein the first end of the frame is secured against a fixed part of the skull, particularly by being strapped to the subject's forehead.

3. An appliance according to claim 1, wherein the pulling platform is centred in front of the face and between the lips.

4. An appliance according to claim 1, wherein the pulling platform is attached to the lower jaw by substantially inextensible means to pull the lower jaw forward.

5. An appliance according to claim 1, wherein the pulling platform includes an adjustment mechanism that is operable to vary the distance by which a subject's mandible is advanced.

6. An appliance according to claim 5, wherein the mechanism is manual or motorised.

7. An appliance according to claim 1, wherein attachment to the lower jaw makes use of orthodontic micro screws or mini or micro implants.

8. An appliance according to claim 1, wherein attachment to the lower jaw makes use of dental prosthetic implants.

9. An appliance according to claim 1, wherein attachment to the lower jaw is made to an appliance that is fixedly or removably secured to the teeth of the lower jaw.

10. An appliance according to claim 1, further comprising an integrated continuous positive airway pressure (CPAP) mask that is connectable with an air source for providing positive airway pressure to a subject.

11. An appliance for human mandibular or maxillary advancement, comprising a frame having:
a first end adapted to be secured against a fixed part of the body of a subject;
a second end equipped with a pulling platform; and
an intervening length to present the pulling platform outside the mouth of the subject when the first end is secured against the fixed part of the body of that subject; wherein, the pulling platform comprises an entirely extra-oral adjustment mechanism to control the distance by which the jaw is advanced when, in use, the mechanism is attached to the subject's jaw.

12. An appliance for human mandibular advancement, comprising a frame to present a pulling platform extra orally centred in front of the face and between the lips of a subject, wherein the pulling platform is attached to the lower jaw to pull the lower jaw forward.

13. An appliance for human mandibular or maxillary advancement, comprising a frame to present a pulling platform extra orally centred in front of the face and between the lips of a subject, wherein the pulling platform is attached to the jaw by substantially inextensible means to pull the jaw forward.

14. A method for advancing the mandible of a human subject, comprising:
locating a pulling platform in a fixed position relative to the subject's head outside the subject's mouth and centred in front of the face and between the lips; and
attaching the pulling platform to the subject's mandible using substantially inextensible means.

15-21. (canceled)
